(19) United States
(12) Patent Application Publication
(10) Pub. No.: US 2025/0359996 A1
(43) Pub. Date: Nov. 27, 2025

(54) MEDICAL IMPLANT INCLUDING POROUS CERAMIC INSERTS FOR THE PROMOTION OF OSSEOINTEGRATION AND FIXATION

(71) Applicant: COORSTEK, INC., Golden, CO (US)

(72) Inventor: Vincent Robert Lucernoni, II, Milford, NH (US)

(21) Appl. No.: 19/211,519

(22) Filed: May 19, 2025

Related U.S. Application Data
(60) Provisional application No. 63/649,983, filed on May 21, 2024.

(51) Int. Cl. A61F 2/36; A61C 8/00; A61F 2/30; A61F 2/34; A61L 27/12; A61L 27/54; A61L 27/56

(52) U.S. Cl. CPC A61F 2/367; A61C 8/0012; A61C 8/0022; A61F 2/30767; A61F 2/3094; A61F 2/34; A61L 27/12; A61L 27/54; A61L 27/56; A61F 2002/30028; A61F 2002/30084; A61F 2002/30224; A61F 2002/30327; A61F 2002/3093; A61F 2002/3401

(57) ABSTRACT

An implant for promoting osseointegration and a method for making the implant are disclosed. The implant includes a first component with a hole extending completely through the first component from a first opening to a second opening and a porous HA insert positioned in and completely filling the hole. The porous HA insert is configured to promote bone growth at the first opening and the second opening.

### CROSS-REFERENCE TO RELATED APPLICATIONS

This application claims the benefit of U.S. Provisional Application No. 63/649,983, entitled, “MEDICAL IMPLANT INCLUDING POROUS CERAMIC INSERTS FOR THE PROMOTION OF OSSEOINTEGRATION AND FIXATION,” filed on May 21, 2024. The entire disclosure of the above application is incorporated herein by reference.

### FIELD

The present disclosure generally relates to metal medical implantables that include porous ceramic inserts for the purpose of improved fixation and osseointegration.

### INTRODUCTION

This section provides background information related to the present disclosure which is not necessarily prior art.

Orthopedic implants, such as femoral rods and acetabular cups for hip surgeries, are formed from a variety of metal, polymer, and/or ceramic materials. Various methods of affixing implants to the patient's existing bone structure exist. These methods include acrylic bone cement, mechanical attachment, and cementless biologic fixation with bone ingrowth. Bone ingrowth generally refers to the formation of bone within a surface of the implant to improve fixation of the implant to the bone. Bone ingrowth may be promoted using various irregular, textured, or porous surfaces of the implant. Surface treatments, such as titanium plasma spray and hydroxyapatite (HA) coatings, have also been used to encourage bone ingrowth with varying degrees of success.

### SUMMARY

In concordance with the instant disclosure, an implant for promoting osseointegration and a method of making an implant is provided. The present technology includes articles of manufacture, systems, and processes that relate to an implant for promoting osscointegration and a method of making an implant.

An implant for promoting osseointegration is disclosed. The implant includes a first component with a hole extending completely through the first component from a first opening to a second opening and a porous HA insert positioned in and completely filling the hole. The porous HA insert is configured to promote bone growth at the first opening and the second opening. In some embodiments, the implant is selected from the group consisting of a femoral rod implant, an acetabular cup implant, a dental implant, and a fixation points for ligament devices.

In some embodiments, the porous HA insert has a substantially cylindrical shape.

In some embodiments, the hole is perpendicular to an axis extending between a proximal end and a distal end of the implant.

In some embodiments, the porous HA insert is affixed to the first component by a press fit, shrink fit, or interference fit.

In some embodiments, the porous HA insert has a porosity of 50% to 85%. In some embodiments, the porous HA insert has macropores each having a size of 10 microns to 500 microns. In some more particular embodiments, an average size of the macropores is between 10 microns to 500 microns. In some more particular embodiments, the porous HA insert has openings interconnecting the macropores, each opening having a size between 10 microns to 500 microns. In some embodiments, the porous HA insert has a compressive strength of at least 6 MPa.

In some embodiments, the implant is the femoral rod implant and the first component is a femoral stem comprising the hole. In some more particular embodiments, the femoral rod implant further comprises a second hole extending completely through the femoral rod implant and a second porous HA insert positioned in and completely filling the second hole. In some even more particular embodiments, the first hole has the same orientation relative to the femoral rod implant as the second hole. In other even more particular embodiments, the first hole has a different orientation relative to the femoral rod implant as the second hole.

In some embodiments, the implant is the acetabular cup implant and the hole extends from an inner surface to an outer surface of the implant. In some more particular embodiments, the implant further comprises a second hole extending from the inner surface to the outer surface of and a second porous HA insert positioned in and completely filling the second hole. In other more particular embodiments, the porous HA insert has a wider portion at the inner surface and a narrower end at the outer surface.

In some embodiments, the implant is a dental implant and the first component is an implant body comprising a screw and the hole.

A method of making an implant for promoting osseointegration is disclosed. The method includes providing a first component of the implant, the first component including a hole extending completely through the first component from a first opening to a second opening, and inserting a porous HA insert into the hole thereby completely filling the hole. The porous HA insert is configured to promote bone growth at the first opening and the second opening.

Further areas of applicability will become apparent from the description provided herein. The description and specific examples in this summary are intended for purposes of illustration only and are not intended to limit the scope of the present disclosure.

### DRAWINGS

The accompanying drawings are incorporated into and form a part of the specification to illustrate several examples of the present disclosure. These drawings, together with the description, explain the principles of the disclosure. The drawings simply illustrate examples of how the disclosure may be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1A is a schematic of an exemplary hydroxyapatite (HA) synthetic bone substitute.

FIG. 1B is an image with 300 micron scale of the exemplary HA synthetic bone substitute of FIG. 1A.

FIG. 1C is an image with 5 micron scale showing an enlarged portion of the image of FIG. 1B.

FIG. 2A is an exemplary femoral rod implant according to a first embodiment of the disclosure.

FIG. 2B is a cross section of a portion of the femoral rod implant of FIG. 2A.

FIG. 3A is an exemplary acetabular cup implant according to a second embodiment of the disclosure.

FIG. 3B is a cross section of a portion of the acetabular cup implant of FIG. 3A.

FIG. 4 is an exemplary dental implant according to a third embodiment of the disclosure.

FIG. 5 is a schematic illustrating an exemplary method of producing an implant according to the present disclosure.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments, including where certain steps can be simultaneously performed, unless expressly stated otherwise. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, all compositional percentages are by weight of the total composition, unless otherwise specified. Disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

Implants, such as femoral rods and acetabular cups used in hip replacements and dental implants, are commonly used in medical procedures. These implants often are affixed to a patient's bone to hold them in place. However, there can be a mismatch in material properties between the implant and bone that prevents the implants from affixing properly. For example, the stiffness of the implant may be greater or less than that of the affixed bone, causing the implant to bend more or less than the nearby bone when a pressure is applied. This in turn may cause bonding, such as bone ongrowth or ingrowth between the implant and affixed bone to crack or otherwise weaken.

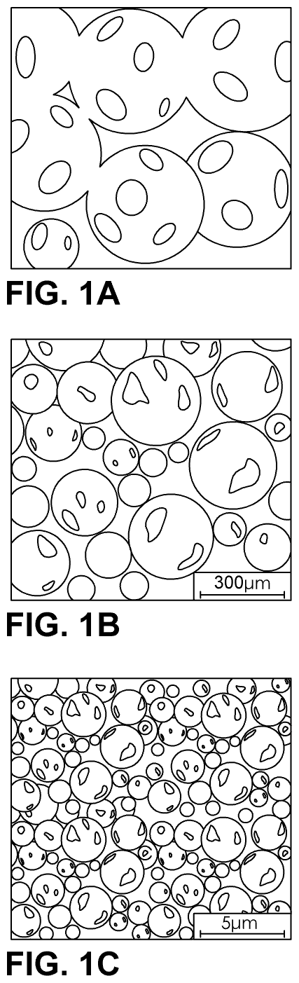

The present disclosure relates generally to implants having an exposed portion of porous hydroxyapatite (HA) to promote bone ingrowth. HA is known to have excellent biocompatibility and to promote bone growth. HA also has a stiffness similar to that of bone material, allowing for better bond retention under stress. An exemplary porous HA, available from CoorsTek KK under the name NEOBONE, is schematically illustrated in FIG. 1A. When sintered, the ceramic porous HA has internal macropores that are interconnected with a series of openings, as shown in the image of FIG. 1B. As shown by the scale in FIG. 1B, these internal interconnected pores are on the order of 10\hat{ }2 microns in size. FIG. 1C shows an enlarged portion of FIG. 1B illustrating the dense strut of the HA material that forms between the internal interconnected pores.

In some embodiments, the porous HA material has a porosity of about 50%, about 55%, about 60%, about 65%, about 70%, about 72%, about 75%, about 78%, about 80%, about 85%, or greater, or within any range defined between any two of the foregoing values, such as 50% to 85%, 70% to 80%, or 72% to 78%.

In some embodiments, the porous HA material has macropores having a size of about 10 microns, 20 microns, 25 microns, 30 microns, 40 microns, 50 microns, 100 microns, 150 microns, 200 microns, 250 microns, 300 microns, 350 microns, 400 microns, 450 microns, 500 microns, or larger, or within any range defined between any two of the foregoing values, such as 10 microns to 500 microns, 30 microns to 400 microns, or 50 microns to 300 microns.

In some embodiments, the porous HA material has openings interconnecting the macropores, the openings having a size of about 10 microns, 20 microns, 25 microns, 30 microns, 40 microns, 50 microns, 100 microns, 150 microns, 200 microns, 250 microns, 300 microns, 350 microns, 400 microns, 450 microns, 500 microns, or larger, or within any range defined between any two of the foregoing values, such as 10 microns to 500 microns, 20 microns to 100 microns, or 30 microns to 50 microns.

In some embodiments, the porous HA material has a compressive strength of at least 6 MPa, at least 7 MPa, at least 7.5 MPa, at least 8 MPa, at least 8.5 MPa, or higher, or within any range defined between any two of the forgoing values, such as 6 MPa to 8.5 MPa or 7.5 MPa to 8.5 MPa.

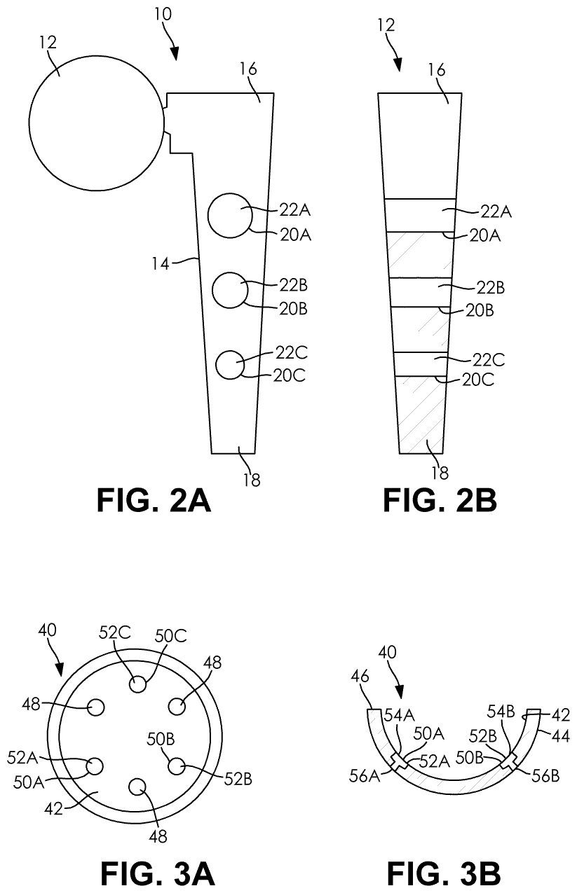

Referring next to FIGS. 2A and 2B, an exemplary femoral rod implant 10 according to a first embodiment of the disclosure is illustrated. Femoral rod implant 10 includes substantially spherical head 12, typically formed form ceramic or metal such as cobalt-chrome, titanium or stainless steel. Head 12 is attached to femoral stem 14, which is driven into the patent's femur as part of a hip replacement operation. Stem 14 is typically formed from a suitable material, such as titanium, cobalt-chromium, stainless steel, and alloys. Stem 14 extends between a proximal end 16 near head 12 and a distal end 18.

One or more holes 20 are provided extending through stem 14. As shown in the cross-section of stem 14 in FIG. 2B, each hole 20 extends all the way through stem 14 perpendicular to an axis of the implant 10 extending between the proximal 16 end and the distal end 18. FIGS. 2A and 2B illustrate three holes 20A, 20B, 20C. In other embodiments, one, two, four, five, or more holes 20 may be present, each extending all the way through stem 14. FIGS. 2A and 2B illustrate all three holes 20A, 20B, 20C in the same orientation relative to implant 10. In other embodiments, one or more of the holes 20 is at a different orientation relative to the other holes 20. For example, one hole 20 may be at an angle of about 30°, about 45°, about 60°, about 90°, about 120°, about 135°, or about 150° to another hole 20. In still other embodiments, each of the holes 20 is at a different orientation relative to the other holes 20, and could be drilled obliquely to the center line. The holes 20 in the stem 14 could be drilled, formed in a casting (lost wax method), lasered, formed through a 3D/Additive Manufacturing methodology, or other method used by those skilled in metalworking and forming technologies.

A porous HA insert 22 is positioned within each hole 20. As illustrated in FIGS. 2A and 2B, each hole 20A, 20B, 20C has a corresponding porous HA insert 22A, 22B, 22C. In some embodiments, each porous HA insert 22 is sized to completely fill the corresponding hole 20. As illustrated in FIG. 2B, each porous HA insert has a substantially cylindrical shape with ends that slope towards each other in the direction of the distal end 18.

As described in more detail below, porous HA inserts 22 may be formed separate from stem 14 and then inserted into the corresponding hole 20 where it is held in place by a press fit, shrink fit, or interference fit. Stem 14 may be heated to expand the size of hole 20 to introduce porous HA insert 22. Upon cooling, the press fit or interference fit between the parts holds porous HA insert 22 in place.

Porous HA inserts 22 promote bone growth into the macropores and interconnecting openings of the porous HA material, which in turn better affixes the insert 22, and more broadly implant 10 to which insert 22 is a part of, to the bone. In some embodiments, porous HA inserts 22 are configured to allow bone growth completely through the porous HA insert 22, securing implant 10.

Referring next to FIGS. 3A and 3B, an exemplary acetabular cup implant 40 according to a second embodiment of the disclosure is illustrated. FIG. 3A illustrates an overhead view looking into the convex inner surface 42 of cup implant 40. As shown in the cross-sectional view of FIG. 3B, cup implant 40 further includes outer surface 44 and rim 46 inner surface 42 and outer surface 44.

Acetabular cup implant 40 may be formed from one or more suitable materials, such as titanium tantalum, alloys, ceramics, or polymers. Cup implant 40 illustratively includes one or more fixation screws 48 for securing implant 40 to the patient's acetabulum.

One or more holes 52 are provided extending through cup implant 40. As shown in the cross-section of FIG. 3B, each hole 52 extends all the way through cup implant 40 from inner surface 42 to outer surface 44. FIGS. 3A and 3B illustrate three holes 52A, 52B, 52C. In other embodiments, one, two, four, five, or more holes 52 may be present, each extending all the way through cup implant 40.

In some embodiments, one or more hole 52 has a substantially equal cross section extending through cup implant 40. In the embodiment illustrated in FIG. 3B, each hole 52A, 52B has a corresponding wider portion 54A, 54B at inner surface 42 and a corresponding narrower portion 56A, 56B at outer surface 44.

A porous HA insert 50 is positioned within each hole 52. As illustrated in FIGS. 3A and 3B, each hole 52A, 52B, 52C, has a corresponding porous HA insert 50A, 50B, 50C. In some embodiments, each porous HA insert 50 is sized to completely fill the corresponding hole 52. As illustrated in FIG. 3B, each porous HA insert 50 has a shape with a larger end corresponding to wider portion 54 at inner surface 42 and a narrower end corresponding to narrower portion 56 at outer surface 44 for each hole 52. This shape helps to retain the insert 50 in place after osseointegration has occurred. As illustrated in FIG. 3B, this shape may be substantially in the shape of a capital “T” letter, although other geometries with a wider portion 54 at inner surface 42, such as a substantially conical shape, may also be used.

As described in more detail below, porous HA inserts 50 may be formed separately and then inserted into the corresponding hole 52 where it is held in place by a press fit or interference fit. Inner surface 42 of implant 40 may be heated to expand the size of hole 52 to introduce porous HA insert 50. Upon cooling, the press fit or interference fit between the parts holds porous HA insert 50 in place.

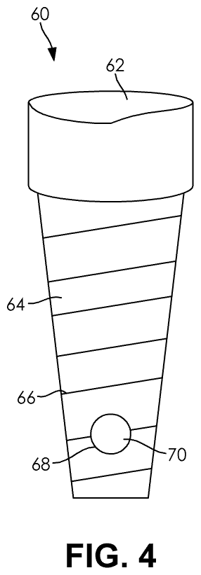

Referring next to FIG. 4, an exemplary dental implant 60 according to a third embodiment of the disclosure is illustrated. Dental implant 60 includes crown 62 attached to implant body 64, made from a metal such as titanium or ceramic such as zirconia. Implant body 64 is secured using screws 66.

One or more holes 68 are providing extending all the way through crown 62 and/or body 64. FIG. 4 illustrates a single hole 68 in implant body 64. In other embodiments, two, three, four, five, or more holes 68 may be present in crown 62, body 64, or a combination thereof, each extending all the way through implant crown 62 or body 64. If multiple holes are present, one or more of the holes 68 may be at a different orientation relative to the other holes 68. For example, one hole 68 may be at an angle of about 0°, about 30°, about 45°, about 60°, about 90°, about 120°, about 135°, or about 150° to another hole 68. In still other embodiments, each of the holes 68 is a different orientation relative to the other holes 68 and could be drilled obliquely to the center line.

A porous HA insert 70 is positioned within each hole 68. As illustrated in FIG. 3, each hole 68A, 68B, 68C has a corresponding porous HA insert 70A, 70B, 70C. In some embodiments, each porous HA insert 70 is sized to completely fill the corresponding hole 68, and each porous HA insert has a substantially cylindrical shape.

As described in more detail below, porous HA inserts 70 may be formed separate from implant body 64 and then inserted into the corresponding hole 68 where it is held in place by a press fit or interference fit. Implant body 64 may be heated to expand the size of hole 68 to introduce porous HA insert 70. Upon cooling, the press fit or interference fit between the parts holds porous HA insert 70 in place.

FIGS. 2A-4 illustrate exemplary femoral rod implant 10, acetabular cup implant 40, and dental implant 60. Porous HA inserts are also contemplated for other implants, such as fixation points for ligament devices. As an alterative or addition to traditional spray-on coatings, these porous HA inserts allow bone to growth through or into the implant to assure a bone-growth anchor into the body.

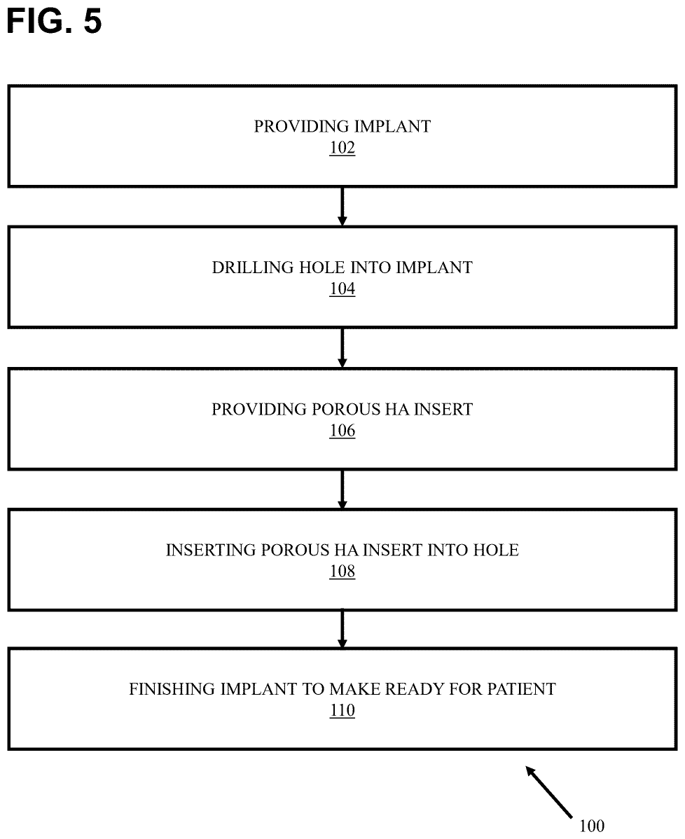

FIG. 5 is a schematic illustrating an exemplary method 100 of producing an implant according to the present disclosure.

In step 102, an implant is provided, such as a femoral rod implant, acetabular cup implant, dental implant, fixation point for ligament device, or other suitable implant.

In step 104, if a hole is not already provided in the implant, a hole is drilled or otherwise formed in the implant.

In step 106, the porous HA insert is provided.

In step 108, the porous HA insert is inserted into the corresponding hole. In some embodiments, a cement or other suitable adhesive is used. In other embodiments, the porous HA insert has a diameter sized to be held in the corresponding insert hole by press fit or interference fit. In some embodiments, at least a portion of the implant proximate the corresponding hole is heated to expand the hole, allowing the porous HA insert to be inserted into the hole.

In step 110, the implant including affixed porous HA insert is finished by machining, polishing, or other suitable process necessary to make it ready for a patient.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

Various modifications of the above-described disclosure will be evident to those skilled in the art. It is intended that such modifications are included within the scope of the following claims.

What is claimed is:

1. An implant for promoting osseointegration, the implant comprising:
a first component with a hole extending completely through the first component from a first opening to a second opening; and
a porous HA insert positioned in and completely filling the hole, the porous HA insert configured to promote bone growth at the first opening and the second opening.

2. The implant of claim 1, wherein the implant includes a member selected from a group consisting of a femoral rod implant, an acetabular cup implant, a dental implant, and a fixation points for ligament devices.

3. The implant of claim 2, wherein the implant is the femoral rod implant and the first component is a femoral stem comprising the hole.

4. The implant of claim 3, wherein the femoral rod implant further comprises a second hole extending completely through the femoral rod implant and a second porous HA insert positioned in and completely filling the second hole.

5. The implant of claim 4, wherein the first hole has a same orientation relative to the femoral rod implant as the second hole.

6. The implant of claim 4, wherein the first hole has a different orientation relative to the femoral rod implant as the second hole.

7. The implant of claim 3, wherein the porous HA insert has a substantially cylindrical shape.

8. The implant of claim 3, wherein the hole is perpendicular to an axis extending between a proximal end and a distal end of the implant.

9. The implant of claim 2, wherein the implant is the acetabular cup implant and the hole extends from an inner surface to an outer surface of the implant.

10. The implant of claim 9, wherein the implant further comprises a second hole extending from the inner surface to the outer surface of and a second porous HA insert positioned in and completely filling the second hole.

11. The implant of claim 9, wherein the porous HA insert has a wider portion at the inner surface and a narrower end at the outer surface.

12. The implant of claim 2, wherein the implant is the dental implant and the first component is an implant body comprising a screw and the hole.

13. The implant of claim 1, wherein the porous HA insert is affixed to the first component by a press fit, a shrink fit, or an interference fit.

14. The implant of claim 1, wherein the porous HA insert has a porosity of 50% to 85%.

15. The implant of claim 1, wherein the porous HA insert has macropores each having a size of 10 microns to 500 microns.

16. The implant of claim 15, wherein an average size of the macropores is between 10 microns to 500 microns.

17. The implant of claim 16, wherein the porous HA insert has openings interconnecting the macropores, each opening having a size between 10 microns to 500 microns.

18. The implant of claim 1, wherein the porous HA insert has a compressive strength of at least 6 MPa.

19. A method of making an implant, the method comprising:
providing a first component of the implant, the first component including a hole extending completely through the first component from a first opening to a second opening; and
inserting a porous HA insert into the hole thereby completely filling the hole, the porous HA insert configured to promote bone growth at the first opening and the second opening.
